(19) United States
(12) Patent Application Publication
(10) Pub. No.: US 2002/0128456 A1
(43) Pub. Date: Sep. 12, 2002

(54) Candida albicans kinase genes and polypeptides and uses thereof

(76) Inventors: Benjamin Stone Amidon, Arlington, MA (US); Christine Ellen Bulawa, Arlington, MA (US)

Correspondence Address: J. Peter Fasse, Fish & Richardson P.C., 225 Franklin Street, Boston, MA, 02110-2804, US

(21) Appl. No.: 09/887,828

(22) Filed: Jun. 22, 2001

Related U.S. Application Data
(60) Provisional application No. 60/213,621, filed on Jun. 23, 2000.

(51) Int. Cl. C07H 21/02; C07H 21/04

(52) U.S. Cl. 536/23.1

(57) ABSTRACT

Disclosed are Candida albicans kinase genes and polypeptides and their use in identifying antifungal agents, for example.

### CROSS REFERENCE TO RELATED APPLICATION

This application claims priority from U.S. Provisional Patent Application No. 60/213,621, filed on Jun. 23, 2000, which is incorporated herein by reference in its entirety.

### FIELD OF THE INVENTION

The invention relates to kinase genes of the fungus Candida albicans and their use in identifying antifungal agents.

### BACKGROUND OF THE INVENTION

Kinases are responsible for phosphorylation of protein substrates, usually via tyrosine, serine, threonine, or other substrates residues of the substrate protein. Since phosphorylation and de-phosphorylation of proteins are common means of modulating protein activity or function, kinases are expected to be involved in the regulation of other proteins.

By way of background, the Fungi Kingdom consists of two divisions, the Eumycota and Myxomycota or the true fungi and slime molds, respectively. The true fungi are those species that are hyphal or are clearly related to species that are hyphal, possess cell walls throughout most or all of their life cycle, and are exclusively absorptive in their function. The slime molds are organisms that do not form hyphae, lack cell walls during the phase in which they obtain nutrients and grow and are capable of ingesting nutrients in particulate form by phagocytosis.

The two most important classes of true fungi in which most species produce motile cells, known as zoospores, are the Oomycetes, and the Chytridiomycetes. The fungi that lack zoospores are classified according to the sexual phase of the fungal life cycle. The sexual process leads to the production of characteristic spores in the different groups. The fungi that form zygospores are classified as Zygomycetes, those that form ascospores are classified as Ascomycetes, and those forming basidiospores are classified as Basidiomycetes. There are also many species, recognizable as higher fungi through the presence of cell walls in their hyphae, that produce asexual spores but lack a sexual phase. These are known as Deuteromycetes, and details of their asexual sporulation are used to classify them. A representative member of the Deuteromycetes includes Candida albicans. These species are extensively reviewed in “The Fungi” (M. J. Carlile and S. C. Watkinson, eds., 1994, Acad Press Ltd.) and “The Growing Fungus” (N. A. R. Gow and G. M. Gadd, eds., 1995, Chapman and Hall).

Yeast are fungi that are normally unicellular and reproduce by budding, although some will, under appropriate conditions, produce hyphae, just as some normally hyphal fungi may produce a yeast phase. The best known of all yeasts is Saccharomyces cerevisiae, which is a member of the Ascomycetes species. It is commonly regarded as a diploid yeast since mating usually soon follows ascospore germination. However, single cells can be used to establish permanently haploid cultures.

Fungal and other mycotic pathogens are responsible for a variety of diseases in humans, animals, and plants. Fungal infection is also a significant problem in veterinary medicine. Some of the fungi that infect animals can be transmitted from animals to humans. Fungal infections or infestations are also a very serious problem in agriculture with fungicides being employed to protect vegetable, fruit, and cereal crops. Fungal attack of wood products is also of major economic importance. Additional products that are susceptible to fungal infestation include textiles, plastics, paper, and paint. Some of these fungal targets are extensively reviewed in WO 95/11969.

Statistics show that the incidence of fungal infections has doubled from the 1980's to the 1990's, and fungal infections of the blood stream have increased fivefold with an observed mortality of 50% (Tally et al., 1997, Int. Conference Biotechnol Microb. Prods.: Novel Pharmacol. Agrobiol. Activities, Williamsburg, Va., Abstract S5, p19). These include fungal infections, such as candidiasis, to which all individuals are susceptible, but also infections such as cryptococcosis and aspergillosis, which occur particularly in patients of compromised immune status.

By way of example, the yeast Candida albicans (C. albicans) has been shown to be one of the most pervasive fungal pathogens in humans. It has the capacity to opportunistically infect a diverse spectrum of compromised hosts, and to invade many diverse tissues in the human body. It can in many instances evade antibiotic treatment and the immune system. Although C. albicans is a member of the normal flora of the mucous membranes in the respiratory, gastrointestinal, and female genital tracts, in such locations it may gain dominance and be associated with pathologic conditions. Sometimes it produces progressive systematic disease in debilitated or immunosuppressed patients, particularly if cell-mediated immunity is impaired. Sepsis may occur in patients with compromised cellular immunity, e.g., those undergoing cancer chemotherapy, or those with lymphoma, AIDS, or other conditions. Candida may produce bloodstream invasion, thrombophlebitis, endocarditis, or infection of the eyes and virtually any organ or tissue when introduced intravenously, e.g., via tubing, needles, narcotics abuse.

Candida albicans has been shown to be diploid with balanced lethals, and therefore probably does not go through a sexual phase or meiotic cycle. This yeast appears to be able to spontaneously and reversibly switch at high frequency between at least seven general phenotypes. Switching has been shown to occur not only in standard laboratory strains, but also in strains isolated from the mouths of healthy individuals.

Nystatin, ketoconazole, and amphotericin B are drugs that have been used to treat oral and systemic Candida infections. However, orally administered nystatin is limited to treatment within the gut and is not applicable to systemic treatment. Some systemic infections are susceptible to treatment with ketoconazole or amphotericin B, but these drugs may not be effective in such treatment unless combined with additional drugs. Amphotericin B has a relatively narrow therapeutic index and numerous undesirable side effects, ranging from nausea and vomiting to kidney damage, and toxicities occur even at therapeutic concentrations. While ketoconazole and other azole antifungals exhibit significantly lower toxicity, their mechanism of action, through inactivation of the cytochrome P₄₅₀ prosthetic group in certain enzymes (some of which are found in humans) precludes use in patients that are simultaneously receiving other drugs that are metabolized by the same enzymes. These adverse effects mean that their use is generally limited to the treatment of topical or superficial infections. In addition, resistance to these compounds is emerging and may pose a serious problem in the future. The more recently developed triazole drugs, such as fluconazole, are believed by some to have fewer side effects but are not completely effective against all pathogens.

Invasive aspergillosis, caused by Aspergillus fumigatus (A. fumigatus) has also become an increasingly opportunistic infection. There has been a 14-fold increase in its incidence during the past 12 years as detected by autopsy, and only two drugs are available that are effective in its treatment, neither of which is completely satisfactory. Amphotericin B must be given intravenously and has a number of toxic side effects. Itraconazole, which can be given orally is often prescribed imprudently, encouraging the emergence of resistant fungal strains (Dunn-Coleman and Prade, Nature Biotechnology, 1998, 16:5). Resistance is also developing to synthetic azoles (such as fluconazole and flucytosine), and the natural polyenes (such as amphotericin B) are limited in use by their toxicity.

Fungicide resistance generally develops when a fungal cell or fungal population that originally was sensitive to a fungicide becomes less sensitive by heritable changes after a period of exposure to the fungicide.

In certain applications, such as agriculture, it is possible to combat resistance through alteration of fungicides or the use of fungicide mixtures. To prevent or delay the build up of a resistant pathogen population, different agents that are effective against a particular disease must be available. One way of increasing the number of available agents is to search for new site-specific inhibitors.

Consequently, antifungal drug discovery efforts have been directed at components of the fungal cell or its metabolism that are unique to fungi, and hence might be used as therapeutic targets of new agents which act on the fungal pathogen without undue toxicity to host cells. Such potential targets include enzymes critical to fungal cell wall assembly (U.S. Pat. No. 5,194,600) as well as topoisomerases (enzymes required for replication of fungal DNA). Two semisynthetic antifungal agents such as the echinocandins and the related pneumocandins are in late stage clinical trials. Both are cyclic lipopeptides produced by fungi that non-competitively inhibit β(1,3)-glucan synthase and thus interfere with the biosynthesis of the fungal cell wall. These clinical candidates are generally more water-soluble, have improved pharmacokinetics and broader antifungal spectra than their natural parent compounds, and have activity spectra that include many Candida species, including C. albicans, and Aspergilli.

Because no single approach may be effective against all fungal pathogens, and because of the possibility of developed resistance to previously effective antifungal compounds, there remains a need for new antifungal agents with novel mechanisms of action and improved or different activity profiles. There is also a need for agents which are active against fungi but are not toxic to mammalian cells, as toxicity to mammalian cells can lead to a low therapeutic index and undesirable side effects in the host (e.g., patient). An important aspect of meeting this need is the selection of an appropriate component of fungal structure or metabolism as a therapeutic target.

Even after a particular intracellular target is selected, the means by which new antifungal agents are identified pose certain challenges. Despite the increased use of rational drug design, a preferred method continues to be the mass screening of compound “libraries” for active agents by exposing cultures of fungal pathogens to the test compounds and assaying for inhibition of growth. In testing thousands or tens of thousands of compounds, however, a correspondingly large number of fungal cultures must be grown over time periods that are relatively long compared to most bacterial culture times. Moreover, a compound that is found to inhibit fungal growth in culture may be acting not on the desired target but on a different, less unique fungal component, with the result that the compound may act against host cells as well and thereby produce unacceptable side effects. Consequently, there is a need for an assay or screening method which more specifically identifies those agents that are active against a certain intracellular target. Additionally, there is a need for assay methods having greater throughput, that is, assay methods that reduce the time and materials needed to test each compound of interest.

Different kinases from C. albicans have been described. For example, a particular histidine kinase gene and a particular protein kinase A gene appear to be involved in controlling the conversion of C. albicans from a unicellular organism to a filamentous organism (Calera et al., Mycoses, 43(suppl 2):49-53, 1999; Sonneborn et al., Mol. Microbiol. 35:386-396, 2000).

### SUMMARY OF THE INVENTION

The invention is based on the discovery of a kinase gene (NRK1) in the fungus Candida albicans, which is essential for survival. Essential genes are genes which are required for growth (such as metabolism, division, or reproduction) and survival of an organism. Essential genes can be used to identify therapeutic antifungal agents. These therapeutic agents can reduce or prevent growth, or decrease pathogenicity or virulence, and preferably, kill the organism.

The C. albicans kinase (CaKinase) coding sequence is depicted in FIG. 1A as SEQ ID NO:1, with the amino acid sequence depicted in FIG. 1B as SEQ ID NO:2. Thus, the present invention relates to a novel kinase enzyme—which is specific to C. albicans—and to a nucleotide sequence (NRK1) encoding the same. The present invention also relates to the use of the novel nucleic acid and amino acid sequences in the diagnosis and treatment of disease. The present invention further relates to the use of the novel nucleic acid and amino acid sequences to evaluate and/or to screen for agents that can modulate kinase activity. In addition, the present invention relates to genetically engineered host cells that include or express the novel nucleic acid and amino acid sequences to evaluate and/or to screen for agents that can modulate kinase activity.

The kinase enzyme of the present invention is obtainable from the C. albicans fungal species.

The kinase enzyme of the present invention may be the same as the naturally occurring form—for this aspect, e.g., the kinase can be encoded by a non-native nucleotide sequence—or a variant, homolog, fragment or derivative thereof. In addition, the kinase is an isolated kinase or purified kinase. The kinase can be obtained from or produced by any suitable source, whether natural or not, or it may be synthetic, semisynthetic, or recombinant.

The kinase gene of the invention is essential for survival of C. albicans. Accordingly, the kinase nucleic acid sequence of the invention, and the kinase polypeptide of the invention, are useful targets for identifying compounds that are inhibitors of C. albicans. Such inhibitors attenuate fungal growth by inhibiting the activity of the kinase polypeptide, or by inhibiting transcription or translation. Accordingly, in one aspect, this invention provides isolated nucleic acid molecules encoding C. albicans kinase polypeptides or biologically active portions thereof, as well as nucleic acid fragments suitable as primers or hybridization probes for the detection of kinase-encoding nucleic acids (e.g., fragments of at least 15 nucleotides (e.g., at least 18, 20, 25, 30, 35, 45, 60, 80, or 100 nucleotides)).

The invention features a nucleic acid molecule that is at least 50% (or 65%, 75%, 85%, 95%, 98%, or 100%) identical to the nucleotide sequence shown in SEQ ID NO:1, or the nucleotide sequence of the cDNA insert of the plasmid deposited with ATCC as Accession Number ______ (the “cDNA of ATCC ______”), or a complement thereof.

The invention features a nucleic acid molecule that includes a fragment of at least 50 (e.g., 100, 150, 200, 300, 325, 350, 375, 400, 425, 450, 500, 550, 600, 650, 700, 800, 900, 1000, 1200, 1400, 1600, 1800, 2000, 2200, or 2400) nucleotides of the nucleotide sequence shown in SEQ ID NO:1, or the nucleotide sequence of the cDNA ATCC ______, or a complement thereof.

The invention also features a nucleic acid molecule that includes a nucleotide sequence encoding a protein having an amino acid sequence that is at least 45% (or 55%, 65%, 75%, 85%, 95%, 98%, or 100%) identical to the amino acid sequence of SEQ ID NO:2 or the amino acid sequence encoded by the cDNA of ATCC ______.

Also within the invention is a nucleic acid molecule that encodes a fragment of a polypeptide having the amino acid sequence of SEQ ID NO:2, the fragment including at least 17 (e.g., 25, 30, 50, 100, 150, 300, 400, or 450) contiguous amino acids of SEQ ID NO:2 or the polypeptide encoded by the cDNA of ATCC Accession Number ______.

In other embodiments, the invention features an isolated kinase protein having an amino acid sequence that is at least about 45% (e.g., 55%, 65%, 75%, 85%, 95%, 98%, or 100%) identical to the amino acid sequence of SEQ ID NO:2; and an isolated kinase protein which is encoded by a nucleic acid molecule having a nucleotide sequence that is at least about 50% (e.g., 60%, 75%, 85%, 95%, or 100%) identical to SEQ ID NO:1 or the cDNA of ATCC ______; and an isolated kinase protein which is encoded by a nucleic acid molecule having a nucleotide sequence which hybridizes under stringent hybridization conditions to a nucleic acid molecule having the nucleotide sequence of SEQ ID NO:1 or the non-coding strand of the cDNA of ATCC ______.

Another embodiment of the invention features kinase nucleic acid molecules that specifically detect C. albicans kinase nucleic acid molecules in a sample containing nucleic acid molecules encoding other kinases. For example, in one embodiment, a C. albicans kinase nucleic acid molecule hybridizes under stringent conditions to a nucleic acid molecule that includes the nucleotide sequence of SEQ ID NO:1, or the cDNA of ATCC ______, or a complement thereof. In another embodiment, the C. albicans kinase nucleic acid molecule is at least 50 (e.g., 100, 200, 300, 400, 500, 700, 900, 1100, or 1300) nucleotides in length and hybridizes under stringent conditions to a nucleic acid molecule that includes the nucleotide sequence shown in SEQ ID NO:1, the cDNA of ATCC ______, or a complement thereof. In another embodiment, the invention provides an isolated nucleic acid molecule that is antisense to the coding strand of a C. albicans kinase nucleic acid.

In another aspect, the invention provides a vector, e.g., a recombinant expression vector, which includes a kinase nucleic acid molecule of the invention. In another embodiment the invention provides a host cell containing such a vector. The invention also provides a method for producing kinase protein by culturing, in a suitable medium, a host cell of the invention containing a recombinant expression vector such that a kinase protein is produced.

Another aspect of this invention features isolated or recombinant kinase proteins and polypeptides. Typical kinase proteins and polypeptides possess at least one biological activity possessed by naturally occurring C. albicans kinase, e.g., an ability to phosphorylate a substrate protein, e.g., on tyrosine, serine, threonine, or histidine residues. It is not necessary that the kinase polypeptide have activity that is equivalent to that of the wild-type C. albicans kinase. For example, the kinase polypeptide can have 20, 50, 75, 90, 100, or an even higher percent of the wild-type activity.

Since the C. albicans kinase gene, which is essential for survival, has been identified, nucleic acids encoding C. albicans kinases can be used to identify antifungal agents. Such antifungal agents can be identified with high throughput assays to detect inhibition of kinase activity. For example, this inhibition can be caused by small molecules binding directly to the kinase polypeptide or by binding of small molecules to other essential polypeptides in a biochemical pathway in which the kinase participates.

The invention also provides methods of identifying agents (such as compounds, substances, or compositions) that affect, or selectively affect, (such as inhibit or otherwise modify) the activity of and/or expression of the kinase, by contacting the kinase or the nucleotide sequence coding for same with the agent and then measuring the activity of the kinase and/or the expression thereof. In a related aspect, the invention features a method of identifying agents (such as compounds, other substances or compositions comprising same) that affect (such as inhibit or otherwise modify) the activity of and/or expression of CaKinase, by measuring the activity of and/or expression of CaKinase in the presence of the agent or after the addition of the agent in: (a) a cell line into which has been incorporated a recombinant construct including the nucleotide sequence of the CaKinase gene (e.g., SEQ ID NO:1) or an allelic variation thereof, or (b) a cell population or cell line that naturally selectively expresses CaKinase, and then measuring the activity of CaKinase and/or the expression thereof.

Since the C. albicans kinase gene described herein has been identified, it can be cloned into various host cells (e.g., fungi, E. coli, or yeast) for carrying out such assays in whole cells. Similarly, conventional in vitro assays of kinase activity can be used with the kinase of the invention.

In one embodiment, the invention features a method for identifying a compound for the treatment of a fungal infection, wherein the method entails, in sequence, (i) preparing a first cell and a second cell, the first and second cells being capable of expressing CaKinase, (ii) contacting the first cell with a test compound, (iii) determining the level of expression of CaKinase in the first and second cells, (iv) comparing the level of expression in the first cell with the second cell, and (v) selecting the test compound for treatment of a fungal infection where expression of CaKinase in the first cell is less than expression of the essential gene in the second cell, and wherein the CaKinase gene is a first nucleic acid molecule which encodes a polypeptide including the amino acid sequence of SEQ ID NO:2, or a naturally occurring allelic variant thereof, and wherein the first nucleic acid molecule hybridizes under stringent conditions to a second nucleic acid molecule, the second nucleic acid molecule consisting of a nucleotide sequence of SEQ ID NO:1. The determination of the level of expression of the CaKinase gene can be made by measuring the amount of mRNA transcribed from the CaKinase gene. Alternatively, the level of CaKinase encoded by the CaKinase gene can be measured.

The test compound can be a small organic or inorganic molecule. Alternatively, the test compound can be a test polypeptide (e.g., a polypeptide having a random or predetermined amino acid sequence; a naturally-occurring or synthetic polypeptide) or a nucleic acid, such as a DNA or RNA molecule; a carbohydrate; or aptamer. The test compound can be a naturally-occurring compound or it can be synthetically produced. Synthetic libraries, chemical libraries, and the like can be screened to identify compounds that bind to the kinase.

In another suitable method, there is provided an assay method for identifying an agent that can affect kinase activity or expression thereof, the assay method comprising contacting an agent with an amino acid sequence or nucleotide sequence according to the present invention; and measuring the activity or expression of the kinase; where a difference in activity between a) kinase activity or expression in the absence of the agent and b) kinase activity or expression in the presence of the agent is indicative that the agent can affect kinase activity or expression.

Another suitable method for identifying antifungal compounds involves screening for small molecules that specifically bind to the new CaKinase. A variety of suitable binding assays are known in the art as described, for example, in U.S. Pat. Nos. 5,585,277 and 5,679,582, incorporated herein by reference. For example, in various conventional assays, test compounds can be assayed for their ability to bind to a polypeptide by measuring the ability of the small molecule to stabilize the polypeptide in its folded, rather than unfolded, state. More specifically, one can measure the degree of protection against unfolding that is afforded by the test compound. Test compounds that bind to a CaKinase with high affinity cause, for example, a significant shift in the temperature at which the polypeptide is denatured. Test compounds that stabilize the polypeptide in a folded state can be further tested for antifungal activity in a standard susceptibility assay.

In a related method for identifying antifungal compounds, a kinase polypeptide is used to isolate peptide or nucleic acid ligands that specifically bind to the kinase polypeptides. These peptide or nucleic acid ligands are then used in a displacement screen to identify small molecules that bind to the kinase polypeptide. Such binding assays can be carried out as described herein.

The CaKinase polypeptides also can be used in assays to identify test compounds that bind to the polypeptides. Test compounds that bind to the kinase polypeptides then can be tested, in conventional assays, for their ability to inhibit fungal growth. Test compounds that bind to the kinase polypeptides are candidate antifungal agents, in contrast to compounds that do not bind to the kinase polypeptides. As described herein, any of a variety of art-known methods can be used to assay for binding of test compounds to the kinase polypeptides.

The invention includes, for example, a method for identifying a compound or candidate compound useful for treating a fungal infection, wherein the method entails (a) measuring the level of expression of the CaKinase gene in a cell in the presence of a test compound; (b) comparing the level of expression measured in step (a) to the level of expression of the CaKinase gene in a cell in the absence of the test compound; and (c) selecting the test compound as being useful for treating a fungal infection when the level of expression of the CaKinase gene in the presence of the test compound is less than the level expression of the CaKinase gene in the absence of the test compound, and wherein the CaKinase gene has the sequence of SEQ ID NO:1. If desired, the level of expression can be measured by measuring the amount of mRNA from the CaKinase gene described herein, or by measuring the amount of protein encoded by the CaKinase gene described herein. Typically, the cell is a C. albicans or Saccharomyces (e.g., Saccharomyces cerevisiae) cell.

In a variation of the above method, the invention features a method for identifying a compound or candidate compound useful for treating a fungal infection, where the method entails (a) measuring the activity of the CaKinase gene in a cell in the presence of a test compound; (b) comparing the activity measured in step (a) to the level activity of the CaKinase gene in a cell in the absence of the test compound; and (c) selecting the test compound as being useful for treating fungal infections when the level of activity of the CaKinase gene measured in the presence of the test compound is less than the level of activity of the CaKinase gene measured in the absence of the test compound, where the CaKinase gene has the sequence of SEQ ID NO:1.

In an alternative method, the invention features a method for identifying a compound or candidate compound useful for treating a fungal infection, where the method entails (a) measuring, in the presence of a test compound, the growth of a sample of cells which have been engineered to express a CaKinase gene; (b) comparing the growth measured in step (a) to the growth of a sample of the cells in the absence of the test compound; and (c) selecting the test compound as being useful for treating a fungal infection when the growth of the sample of cells in the presence of the test compound is slower than the growth of a sample of cells in the absence of the test compound, where the CaKinase gene has the sequence of SEQ ID NO:1. Typically, the cell sample contains fungal cells (e.g., C. albicans).

The invention also includes a method for identifying an antifungal agent where the method entails: (a) contacting a CaKinase polypeptide with a test compound; (b) detecting binding of the test compound to the polypeptide; and (c) determining whether a test compound that binds to the polypeptide inhibits growth of C. albicans, relative to growth of fungi cultured in the absence of the test compound, as an indication that the test compound is an antifungal agent. If desired, the test compound can be immobilized on a substrate, and binding of the test compound to CaKinase is detected as immobilization of CaKinase on the immobilized test compound. Immobilization of CaKinase on the test compound can be detected in an immunoassay with an antibody that specifically binds to CaKinase.

In still another method, binding of a test compound to a kinase polypeptide can be detected in a conventional two-hybrid system for detecting protein/protein interactions (e.g., in yeast or mammalian cells). A test compound found to bind to CaKinase can be further tested for antifungal activity in a conventional susceptibility assay. Generally, in such two-hybrid methods, (a) CaKinase is provided as a fusion protein that includes the polypeptide fused to (i) a transcription activation domain of a transcription factor or (ii) a DNA-binding domain of a transcription factor; (b) the test polypeptide is provided as a fusion protein that includes the test polypeptide fused to (i) a transcription activation domain of a transcription factor or (ii) a DNA-binding domain of a transcription factor; and (c) binding of the test polypeptide to the polypeptide is detected as reconstitution of a transcription factor. Reconstitution of the transcription factor can be detected, for example, by detecting transcription of a gene that is operably linked to a DNA sequence bound by the DNA-binding domain of the reconstituted transcription factor (See, for example, White, 1996, Proc. Natl. Acad. Sci. 93:10001-10003 and references cited therein and Vidal et al., 1996, Proc. Natl. Acad. Sci. 93:10315-10320).

In an alternative method, an isolated nucleic acid molecule encoding a kinase is used to identify a compound that decreases the expression of kinase in vivo (i.e., in a C. albicans cell). Such compounds can be used as antifungal agents. To discover such compounds, cells that express a kinase are cultured, exposed to a test compound (or a mixture of test compounds), and the level of kinase expression or activity is compared with the level of kinase expression or activity in cells that are otherwise identical but that have not been exposed to the test compound(s). Standard quantitative assays of gene expression and kinase activity can be utilized in this aspect of the invention.

To identify compounds that modulate expression of the CaKinase the test compound(s) can be added at varying concentrations to the culture medium of C. albicans. Such test compounds can include small molecules (typically, non-protein, non-polysaccharide chemical entities), polypeptides, and nucleic acids. The expression of the kinase is then measured, for example, by Northern blot PCR analysis or RNAse protection analyses using a nucleic acid molecule of the invention as a probe. The level of expression in the presence of the test molecule, compared with the level of expression in its absence, will indicate whether or not the test molecule alters the expression of CaKinase. Because the CaKinase is essential for survival, test compounds that inhibit the expression and/or function of the kinase will inhibit growth of, or kill, the cells that express the kinase.

More generally, binding of a test compound to a kinase polypeptide can be detected either in vitro or in vivo. If desired, the above-described methods for identifying compounds that modulate the expression of the kinase polypeptides of the invention can be combined with measuring the levels of kinase expressed in cells, e.g., by carrying out an assay of kinase activity, as described above or, for example, performing a Western blot analysis using antibodies that bind to the kinase. The antifungal agents identified by the methods of the invention can be used to inhibit a wide spectrum of pathogenic or nonpathogenic fungal strains.

The invention also features a method for identifying an antifungal agent, where the method entails (a) contacting an CaKinase polypeptide with a test compound; (b) detecting a decrease in activity of CaKinase contacted with test compound; (c) selecting as a candidate compound useful for treating a fungal infection one that decreases the activity of CaKinase; and, optionally, (d) determining whether a candidate compound that decreases activity of a contacted CaKinase polypeptide inhibits growth of fungi, relative to growth of fungi cultured in the absence of the candidate compound that decreases activity of a contacted kinase polypeptide, where inhibition of growth indicates that the candidate compound is an antifungal agent, and where CaKinase is encoded by a gene having the sequence of SEQ ID NO:1. The test compound can be, without limitation, a polypeptide, ribonucleic acid, small molecule, deoxyribonucleic acid, antisense oligonucleotide, or ribozyme.

In yet another embodiment, the invention features a method for identifying a candidate compound that may be useful for treating a fungal infection, wherein the method entails (a) contacting a variant, homolog, or ortholog of a CaKinase polypeptide with a test compound; (b) detecting binding of the test compound to the variant, homolog, or ortholog of CaKinase; and (c) selecting as a candidate compound one that binds to the variant, homolog, or ortholog of CaKinase, wherein CaKinase is encoded by a gene having the sequence of SEQ ID NO:1. Optionally, the method can also include (d) determining whether a candidate compound that binds to the variant, homolog, or ortholog of CaKinase inhibits growth of fungi, relative to growth of fungi cultured in the absence of the candidate, where inhibition of growth indicates that the candidate compound is an antifungal agent. The variant, homolog, or ortholog can be derived from a non-pathogenic or pathogenic fungus.

Some specific embodiments of the present invention relate to assay methods for the identification of antifungal agents using assays for antifungal agents which may be carried out both in whole cell preparations and in ex vivo cell-free systems. In each instance, the assay target is the CaKinase nucleotide sequence—which is essential for fungal viability—and/or the kinase polypeptide. Candidate agents which are found to inhibit the target nucleotide sequence and/or CaKinase in any assay method of the present invention are thus identified as potential or candidate antifungal agents. It is expected that the assay methods of the present invention will be suitable for both small and large-scale screening of test compounds as well as in quantitative assays such as serial dilution studies where the target kinase nucleotide sequence or the kinase polypeptide are exposed to a range of candidate agent concentrations.

When the assay methods of the present invention are carried out as a whole-cell assay, the target kinase nucleotide sequence and/or the kinase polypeptide and the entire living fungal cell may be exposed to the candidate agent under conditions normally suitable for growth. Optimal conditions including essential nutrients, optimal temperatures and other parameters, depending upon the particular fungal strain and suitable conditions being used, are well known in the art. Inhibition of expression of the target nucleotide sequence and/or the activity of CaKinase may be determined in a number of ways including observing the cell culture's growth or lack thereof. Such observation may be made visually, by optical densitometric or other light absorption/scattering means, or by yet other suitable means, whether manual or automated.

In the above whole-cell assay, an observed lack of cell growth may be due to inhibition of the target nucleotide sequence and/or CaKinase or may be due to an entirely different effect of the candidate agent, and further evaluation may be required to establish the mechanism of action and to determine whether the candidate agent is a specific inhibitor of the target. Accordingly, and in one embodiment of the present invention, the method may be performed as a paired-cell assay in which each test compound is separately tested against two different fungal cells, the first fungal cells having a target with altered properties that make it more susceptible to inhibition compared with that of the second fungal cells.

One manner of achieving differential susceptibility is by using mutant strains expressing a modified target kinase polypeptide. A particularly useful strain is one having a temperature sensitive (“ts”) mutation as a result of which the target is more prone than the wild type target to loss of functionality at high temperatures (that is, temperatures higher than optimal, but still permitting growth in wild type cells). When grown at semi-permissive temperatures, the activity of a ts mutant target may be attenuated but sufficient for growth.

Alternatively or in conjunction with the above, differential susceptibility to target inhibitors may be obtained by using a second fungal cell that has altered properties that make it less susceptible to inhibition compared with that of wild type cells such as, for example, a fungal cell that has been genetically manipulated to cause overexpression of a target of the inhibitor. Such overexpression can be achieved by placing into a wild type cell a plasmid carrying the nucleotide sequence for the target. The techniques for generating temperature sensitive mutants, for preparing specific plasmids, and for transforming cell lines with such plasmids are well known in the art.

Alternatively or in conjunction with the above, the access of candidate agents to a cell or an organism may be enhanced by mutating or deleting a gene or genes that encode a protein or proteins responsible for providing a permeability barrier for a cell or an organism.

The present invention also relates to a method for identifying antifungal agents utilizing fungal cell systems that are sensitive to perturbation to one or several transcriptional/translational components.

By way of example, the present invention relates to a method of constructing mutant fungal cells in which one or more of the transcriptional/translational components is present in an altered form or in a different amount compared with a corresponding wild-type cell. This method further involves examining a candidate agent for its ability to perturb transcription/translation by assessing the impact it has on the growth of the mutant and wild-type cells. Agents that perturb transcription/translation by acting on a particular component that participates in transcription/translation may cause a mutant fungal cell which has an altered form or amount of that component to grow differently from the corresponding wild-type cell, but do not affect the growth relative to the wild type cell of other mutant cells bearing alterations in other components participating in transcription/translation. This method thus provides not only a means to identify whether a candidate agent perturbs transcription/translation but also an indication of the site at which it exerts its effects. The transcriptional/translational component which is present in altered form or amount in a cell whose growth is affected by a candidate agent is likely to be the site of action of the agent.

By way of example, the present invention provides a method for identifying antifungal agents which interfere with steps in translational accuracy, such as maintaining a proper reading frame during translation and terminating translation at a stop codon. This method involves constructing mutant fungal cells in which a detectable reporter polypeptide can only be produced if the normal process of staying in one reading frame or of terminating translation at a stop codon has been disrupted. This method further involves contacting the mutant fungal cells with a candidate agent to examine whether it increases or decreases the production of the reporter polypeptide.

The present invention also provides a method of screening an agent for specific binding affinity with CaKinase (or a derivative, homolog, variant or fragment thereof) or the nucleotide sequence coding for same (including a derivative, homolog, variant or fragment thereof), the method comprising the steps of: a) providing a candidate agent; b) combining CaKinase (or the derivative, homolog, variant or fragment thereof) or the nucleotide sequence coding for same (or the derivative, homolog, variant or fragment thereof) with the candidate agent for a time sufficient to allow binding under suitable conditions; such binding or interaction being associated with a second component capable of providing a detectable signal in response to the binding or interaction of the kinase polypeptide or the nucleotide sequence encoding same with the agent; and c) determining whether the agent binds to or otherwise interacts with and activates or inhibits an activity of CaKinase (or the derivative, homolog, variant or fragment thereof) or the expression of the nucleotide sequence coding for same (or the derivative, homolog, variant or fragment thereof) by detecting the presence or absence of a signal generated from the binding and/or interaction of the agent with CaKinase (or the derivative, homolog, variant or fragment thereof) or the nucleotide sequence coding for same (or the derivative, homolog, variant or fragment thereof).

In other embodiments, the cell system is an extract of a fungal cell that is grown under defined conditions, and the method involves measuring transcription or translation in vitro. Such defined conditions are selected so that transcription or translation of the reporter is increased or decreased by the addition of a transcription inhibitor or a translation inhibitor to the cell extract.

One such method for identifying antifungal agents relies upon a transcription-responsive gene product. This method involves constructing a fungal cell in which the production of a reporter molecule, measured as a percentage of over-all transcription, increases or decreases under conditions in which overall fungal cell transcription is reduced. Specifically, the reporter molecule is encoded by a nucleic acid transcriptionally linked to a sequence constructed and arranged to cause a relative increase or decrease in the production of the reporter molecule when overall transcription is reduced. Typically, the overall transcription is measured by the expression of a second indicator gene whose expression, when measured as a percentage of overall transcription, remains constant when the overall transcription is reduced. The method further involves contacting the fungal cell with a candidate agent, and determining whether the agent increases or decreases the production of the first reporter molecule in the fungal cell.

In one embodiment, the reporter molecule is itself the transcription-responsive gene product whose production increases or decreases when overall transcription is reduced. In another embodiment, the reporter is a different molecule whose production is linked to that of the transcription-responsive gene product. Such linkage between the reporter and the transcription-responsive gene product can be achieved in several ways. A gene sequence encoding the reporter may, for example, be fused to part or all of the gene encoding the transcription-responsive gene product and/or to part or all of the genetic elements that control the production of the gene product. Alternatively, the transcription-responsive gene product may stimulate transcription of the gene encoding the reporter, either directly or indirectly.

Alternatively, the method for identifying antifungal agents relies upon a translation-responsive gene product. This method involves constructing a fungal cell in which the production of a reporter molecule, measured as a percentage of over-all translation, increases or decreases under conditions in which overall fungal cell translation is reduced. Specifically, the reporter molecule is encoded by nucleic acid either translationally linked or transcriptionally linked to a sequence constructed and arranged to cause a relative increase or decrease in the production of the reporter molecule when overall translation is reduced. Typically, the overall translation is measured by the expression of a second indicator gene whose expression, when measured as a percentage of overall translation, remains constant when the overall translation is reduced. The method further involves contacting the fungal cell with a candidate agent, and determining whether the agent increases or decreases the production of the first reporter molecule in the fungal cell.

In one embodiment, the reporter molecule is itself the translation-responsive gene product whose production increases or decreases when overall translation is reduced. In another embodiment, the reporter is a different molecule whose production is linked to that of the translation-responsive gene product. Such linkage between the reporter and the translation-responsive gene product can be achieved in several ways. A gene sequence encoding the reporter may, for example, be fused to part or all of the gene encoding the translation-responsive gene product and/or to part or all of the genetic elements that control the production of the gene product. Alternatively, the translation-responsive gene product may stimulate translation of the gene encoding the reporter, either directly or indirectly.

Generally, a wide variety of reporters may be used, with typical reporters providing conveniently detectable signals (e.g., by spectroscopy). By way of example, a reporter gene may encode an enzyme which catalyses a reaction that alters light absorption properties.

Examples of reporter molecules include but are not limited to β-galactosidase, invertase, green fluorescent protein, luciferase, chloramphenicol, acetyltransferase, beta-glucuronidase, exo-glucanase and glucoamylase. Alternatively, radiolabeled or fluorescent tag-labeled nucleotides can be incorporated into nascent transcripts that are then identified when bound to oligonucleotide probes. For example, the production of the reporter molecule can be measured by the enzymatic activity of the reporter gene product, such as β-galactosidase.

In another embodiment of the present invention, a selection of hybridization probes corresponding to a predetermined population of genes of the selected fungal organism may be used to specifically detect changes in gene transcription which result from exposing the selected organism or cells thereof to a candidate agent. In this embodiment, one or more cells derived from the organism is exposed to the candidate agent in vivo or ex vivo under conditions wherein the agent effects a change in gene transcription in the cell to maintain homeostasis. Thereafter, the gene transcripts, primarily mRNA, of the cell or cells are isolated by conventional means. The isolated transcripts or cDNAs complementary thereto are then contacted with an ordered matrix of hybridization probes, each probe being specific for a different one of the transcripts, under conditions where each of the transcripts hybridizes with a corresponding one of the probes to form hybridization pairs. The ordered matrix of probes provides, in aggregate, complements for an ensemble of genes of the organism sufficient to model the transcriptional responsiveness of the organism to a candidate agent. The probes are generally immobilized and arrayed onto a solid substrate such as a microtiter plate. Specific hybridization may be effected, for example, by washing the hybridized matrix with excess non-specific oligonucleotides. A hybridization signal is then detected at each hybridization pair to obtain a transcription signal profile. A wide variety of hybridization signals may be used. In one embodiment, the cells are pre-labeled with radionucleotides such that the gene transcripts provide a radioactive signal that can be detected in the hybridization pairs. The transcription signal profile of the agent-treated cells is then compared with a transcription signal profile of negative control cells to obtain a specific transcription response profile to the candidate agent.

A variety of protocols for detecting and measuring the expression of CaKinase, using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on CaKinase polypeptides is suitable; alternatively, a competitive binding assay may be employed. These and other assays are described, among other places, in Hampton, R et al. (1990, Serological Methods, A Laboratory Manual, APS Press, St Paul, Minn.) and Maddox, D E et al. (1983, J. Exp. Med. 158:121).

In an embodiment of the present invention, CaKinase or a variant, homolog, fragment or derivative thereof and/or a cell line that expresses CaKinase or variant, homolog, fragment or derivative thereof may be used to screen for antibodies, peptides, or other agents, such as organic or inorganic molecules, that act as modulators of CaKinase activity, thereby identifying a therapeutic agent capable of modulating the activity of CaKinase. For example, antibodies that specifically bind to a kinase polypeptide and are capable of neutralizing the activity of CaKinase may be used to inhibit CaKinase activity. Alternatively, screening of peptide libraries or organic libraries made by combinatorial chemistry with recombinantly expressed kinase polypeptide or a variant, homolog, fragment or derivative thereof or cell lines expressing CaKinase or a variant, homolog, fragment or derivative thereof may be useful for identification of therapeutic agents that function by modulating CaKinase activity. Synthetic compounds, natural products, and other sources of potentially biologically active materials can be screened in a number of ways deemed to be routine to those of skill in the art. For example, nucleotide sequences encoding the N-terminal region of CaKinase can be expressed in a cell line and used for screening of allosteric modulators, either agonists or antagonists, of CaKinase activity.

Accordingly, the present invention provides a method for screening a plurality of agents for specific binding affinity with CaKinase, or a portion, variant, homolog, fragment or derivative thereof, by providing a plurality of agents; combining CaKinase or a portion, variant, homolog, fragment or derivative thereof with each of a plurality of agents for a time sufficient to allow binding under suitable conditions; and detecting binding of CaKinase, or portion, variant, homolog, fragment or derivative thereof, to each of the plurality of agents, thereby identifying the agent or agents which specifically bind CaKinase. In such an assay, the plurality of agents may be produced by combinatorial chemistry techniques known to those of skill in the art.

Another technique for screening provides for high throughput screening of agents having suitable binding affinity to CaKinase polypeptides and is based upon the method described in detail in WO 84/03564. In summary, large numbers of different small peptide test compounds are synthesized on a solid substrate, such as plastic pins or some other surface. The peptide test agents are reacted with CaKinase fragments and washed. A bound kinase polypeptide is then detected—such as by appropriately adapting methods well known in the art. A purified kinase polypeptide can also be coated directly onto plates for use in the aforementioned drug screening techniques. Alternatively, non-neutralizing antibodies can be used to capture the peptide and immobilize it on a solid support.

Typically, in an antifungal discovery process, potential new antifungal agents are tested for their ability to inhibit the in vitro activity of the purified expression product of the present invention in a biochemical assay. Agents with inhibitory activity can then progress to an in vitro antifungal activity screening using a standard MIC (Minimum Inhibitory Concentration) test (based on the M27-A NCCLS approved method). Antifungal active agents identified at this point are then tested for antifungal efficacy in vivo, such as by using rodent systemic candidiasis/aspergillosis models. Efficacy is measured by measuring the agent's ability to increase the host animal's survival rate against systemic infection, and/or reduce the fungal burden in infected tissues, compared to control animals receiving no administered agent (which can be by oral or intravenous routes).

The present invention also provides a pharmaceutical composition for treating an individual in need of such treatment of a disease caused by C. albicans (or that can be treated by inhibiting CaKinase activity); the treatment method entails administering a therapeutically effective amount of an agent that affects (such as inhibits) the activity and a pharmaceutically acceptable carrier, diluent, excipient, or adjuvant.

The pharmaceutical compositions can be used for humans or animals and will typically include any one or more of a pharmaceutically acceptable diluent, carrier, excipient or adjuvant. The choice of pharmaceutical carrier, excipient, or diluent can be selected with regard to the intended route of administration and standard pharmaceutical practice. The pharmaceutical compositions can include as (or in addition to) the carrier, excipient, or diluent, any suitable binder(s), lubricant(s), suspending agent(s), coating agent(s), or solubilizing agent(s).

The invention includes pharmaceutical formulations that include a pharmaceutically acceptable excipient and an antifungal agent identified using the methods described herein. In particular, the invention includes pharmaceutical formulations that contain antifungal agents that inhibit the growth of, or kill, pathogenic fungal strains (e.g., pathogenic Candida albicans strains). Such pharmaceutical formulations can be used in a method of treating a fungal infection in an organism. Such a method entails administering to the organism a therapeutically effective amount of the pharmaceutical formulation, i.e., an amount sufficient to ameliorate signs and/or symptoms of the fungal infection. In particular, such pharmaceutical formulations can be used to treat fungal infections in mammals such as humans and domesticated mammals (e.g., cows, pigs, dogs, and cats), and in plants. The efficacy of such antifungal agents in humans can be estimated in an animal model system well known to those of skill in the art (e.g., mouse systems of fungal infections).

The invention also includes (i) a method of treating a mycosal and/or fungal infection in a target (which target can be a living organism, such as a mammal, such as a human, or an inanimate target, such as a textile piece, paper, plastic etc.), which method entails delivering (such as administering or exposing) an effective amount of an agent capable of modulating the expression pattern of the nucleotide sequence of the present invention or the activity of the expression product thereof; and (ii) a method of treating a mycosal and/or fungal infection in a target (which target can be a living organism, such as a plant or a mammal, such as a human, or an inanimate target, such as a textile piece, paper, plastic, etc.), which method entails delivering (such as administering or exposing) an effective amount of an agent identified by an assay according to the present invention. As used herein, the terms “treating,” “treat,” or “treatment” include, inter alia, preventative (e.g., prophylactic), palliative, and curative treatment of fungal infections.

The invention also features a method for inducing an immunological response in an individual, particularly a mammal, which entails inoculating the individual with one or more of the kinase genes or polypeptides described herein, and generally in an amount adequate to produce an antibody and/or T cell immune response to protect the individual from mycoses, fungal infection, or infestations. In another aspect, the present invention relates to a method of inducing an immunological response in an individual which entails delivering to the individual a vector that includes a kinase gene described herein or a variant, homolog, fragment, or derivative thereof in vivo to induce an immunological response, such as to produce antibody and/or a T-cell immune response to protect the individual from disease whether that disease is already established within the individual or not.

Various affinity reagents that are permeable to the microbial membrane (i.e., antibodies and antibody fragments) are useful in practicing the methods of the invention. For example polyclonal and monoclonal antibodies that specifically bind to the C. albicans kinase polypeptide can facilitate detection of C. albicans kinase in various fungal strains (or extracts thereof). These antibodies also are useful for detecting binding of a test compound to the kinase (e.g., using the assays described herein). In addition, monoclonal antibodies that specifically bind to C. albicans kinase can themselves be used as antifungal agents.

In another aspect, the invention features a method for detecting a C. albicans kinase polypeptide in a sample. This method includes: obtaining a sample suspected of containing a C. albicans kinase polypeptide; contacting the sample with an antibody that specifically binds to a C. albicans kinase polypeptide under conditions that allow the formation of complexes of the antibody and the kinase polypeptide; and detecting the complexes, if any, as an indication of the presence of a C. albicans kinase polypeptide in the sample.

In all of the foregoing methods, homologs, orthologs, or variants of the kinase genes and polypeptides described herein can be substituted. While “homologs” are structurally similar genes contained within a species, “orthologs” are functionally equivalent genes from other species (within or outside of a given genus, e.g., from E. coli). The terms “variant,” “homolog,” or “fragment” in relation to the amino acid sequence of the kinase of the invention include any substitution, variation, modification, replacement, deletion, or addition of one or more amino acids from or to the sequence providing the resultant kinase polypeptide.

The invention offers several advantages. The invention provides targets, based on essential functions, for identifying potential agents for the effective treatment of opportunistic infections caused by C. albicans and other related fungal species. Also, the methods for identifying antifungal candidates or agents can be configured for high throughput screening of numerous candidate antifungal agents. Because the kinase gene disclosed herein is thought to be highly conserved, antifungal drugs targeted to this gene or its gene products are expected to have a broad spectrum of antifungal activity.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein. All publications, patent applications, patents, and other references mentioned herein are incorporated herein by reference in their entirety. In the case of a conflict, the present specification, including definitions, will control. In addition, the materials, methods, and examples are illustrative and are not intended to limit the scope of the invention, which is defined by the claims.

Other features and advantages of the invention will be apparent from the following detailed description, and from the claims.

### BRIEF DESCRIPTION OF THE DRAWING

FIGS. 1A and 1B are listings of the nucleotide sequence (SEQ ID NO:1; FIG. 1A) and predicted amino acid sequence (SEQ ID NO:2; FIG. 1B) of Candida albicans kinase (CaKinase) gene NRK1.

### DETAILED DESCRIPTION OF THE INVENTION

A gene encoding kinase of Candida albicans has been identified and is essential for the survival of C. albicans. The kinase gene and polypeptide are useful targets for identifying compounds that are, or potentially are, inhibitors of the fungi in which kinase polypeptides are expressed.

Nucleic acids described herein include both RNA and DNA, including genomic DNA and synthetic (e.g., chemically synthesized) DNA. Nucleic acids can be double-stranded or single-stranded. Where single-stranded, the nucleic acid can be a sense strand or an antisense strand. Nucleic acids can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

An isolated nucleic acid is a DNA or RNA that is not immediately contiguous with both of the coding sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to the coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a genomic DNA fragment produced by PCR or restriction endonuclease treatment) independent of other sequences. It also includes a recombinant DNA that is part of a hybrid gene encoding an additional polypeptide sequence. The terms “isolated” and “purified” refer to a nucleic acid or polypeptide that is substantially free of cellular or viral material with which it is naturally associated, or culture medium (when produced by recombinant DNA techniques), or chemical precursors or other chemicals (when chemically synthesized). Moreover, an isolated nucleic acid fragment is a nucleic acid fragment that is not naturally occurring as a fragment and would not be found in the natural state.

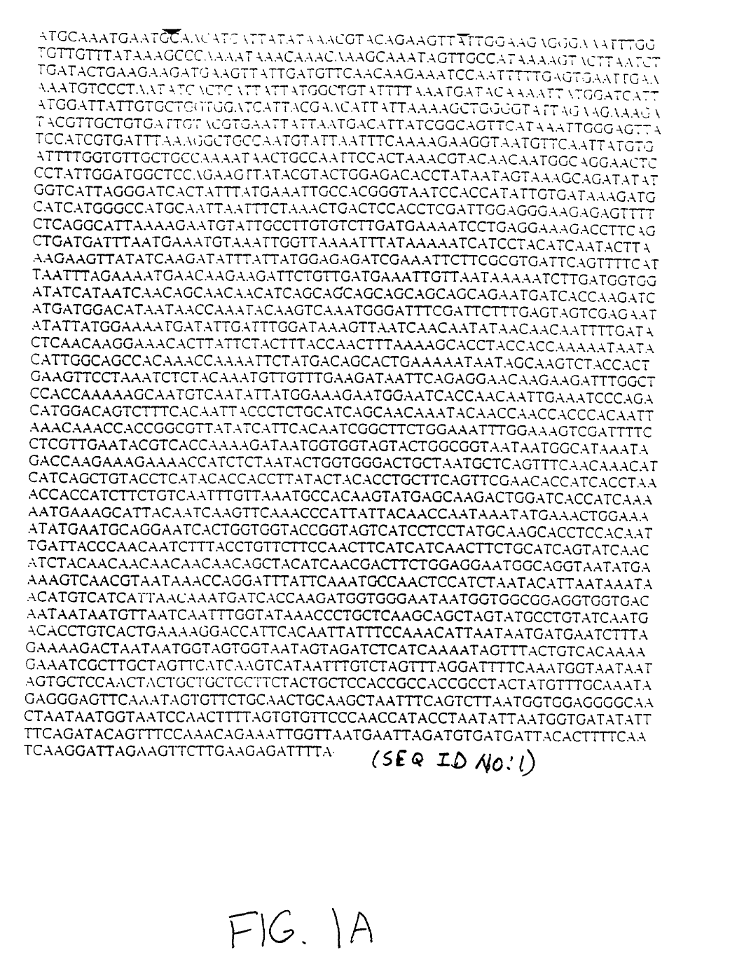

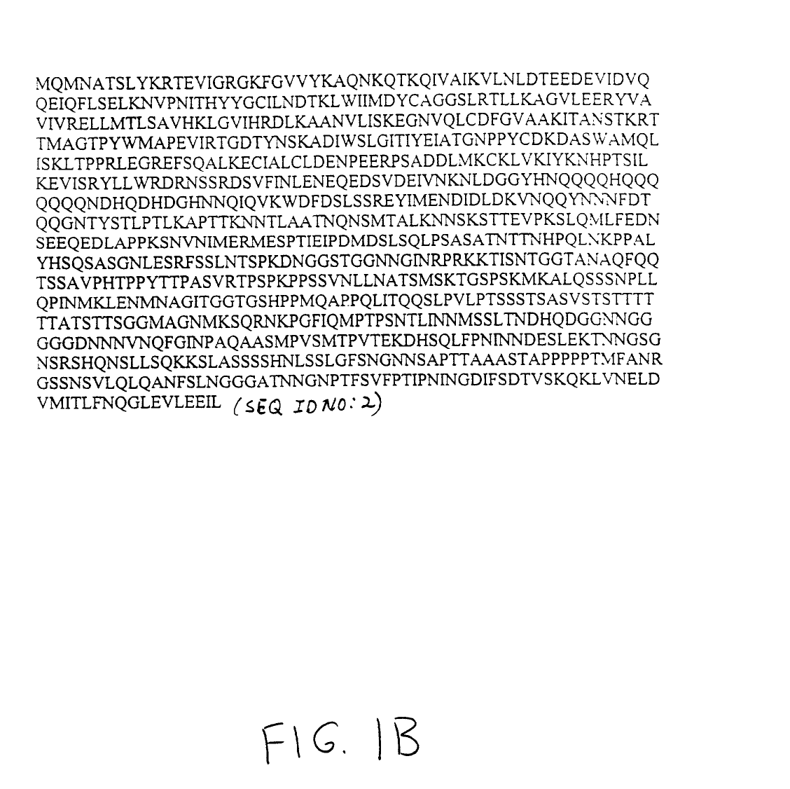

A nucleic acid sequence that is substantially identical to a kinase nucleotide sequence is at least 80% identical to the nucleotide sequence of kinase as represented by SEQ ID NO:1, as depicted in FIG. 1B. For purposes of comparison of nucleic acids, the length of the reference nucleic acid sequence will generally be at least 40 nucleotides, e.g., at least 60 or more nucleotides.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=#of identical positions/total # of overlapping positions×100). Preferably, the two sequences are the same length.

The determination of percent identity or homology between two sequences can be accomplished using a mathematical algorithm. A preferred, non-limiting example of a mathematical algorithm utilized for the comparison of two sequences is the algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to kinase nucleic acid molecules of the invention. BLAST protein searches can be performed with the XBLAST program, score=50, wordlength=3 to obtain amino acid sequences homologous to kinase protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See, e.g., http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted. For purposes of amino acid sequence comparison, the length of a reference kinase polypeptide sequence will generally be at least 16 amino acids, e.g., at least 20 or 25 amino acids.

The terms “variant,” “homolog,” or “fragment” in relation to the nucleotide sequence encoding CaKinase of the present invention include any substitution, variation, modification, replacement, deletion, or addition of one (or more) nucleotides from or to the sequence of a kinase gene. Typically, the resultant nucleotide sequence encodes or is capable of encoding a kinase polypeptide that generally is at least as biologically active as the referenced kinase polypeptide (e.g., as represented by SEQ ID NO:2). In particular, the term “homolog” covers homology with respect to structure and/or function providing the resultant nucleotide sequence codes for or is capable of coding for a kinase polypeptide being at least as biologically active as CaKinase encoded by the sequence shown as SEQ ID NO:1. With respect to sequence homology, there is at least 50% (e.g., 60%, 75%, 85%, 90%, 95%, 98%, or 100%) homology to the sequence shown as SEQ ID NO:1. The term “homology” as used herein can be equated with the term “identity”. Relative sequence homology (i.e., sequence identity) can be determined by commercially available computer programs that can calculate the percent homology between two or more sequences. A typical example of such a computer program is CLUSTAL.

“Substantial identity” means at least 80% sequence identity, as judged by direct sequence alignment and comparison. “Substantial identity” when assessed by the BLAST algorithm equates to sequences which match with an EXPECT value of at least about 7, e.g., at least about 9, 10, or more. The default threshold for EXPECT in BLAST searching is usually 10.

Also included within the scope of the present invention are alleles of CaKinase gene. As used herein, an “allele” or “allelic sequence” is an alternative form of CaKinase. Alleles result from a mutation, i.e., a change in the nucleotide sequence, and generally produce altered mRNAs or polypeptides whose structure or function may or may not be altered. Any given gene can have none, one, or more than one allelic form. Common mutational changes that give rise to alleles are generally ascribed to deletions, additions, or substitutions of amino acids. Each of these types of changes can occur alone, or in combination with the others, one or more times in a given sequence.

The kinase polypeptides of the invention include, but are not limited to, recombinant polypeptides and natural polypeptides. Also included are nucleic acid sequences that encode forms of kinase polypeptides in which naturally occurring amino acid sequences are altered or deleted. Preferred nucleic acids encode polypeptides that are soluble under normal physiological conditions. Also within the invention are nucleic acids encoding fusion proteins in which a portion of the kinase polypeptide is fused to an unrelated polypeptide (e.g., a marker polypeptide or a fusion partner) to create a fusion protein. For example, the polypeptide can be fused to a hexa-histidine tag to facilitate purification of bacterially expressed polypeptides, or to a hemagglutinin tag to facilitate purification of polypeptides expressed in eukaryotic cells. The invention also includes, for example, isolated polypeptides (and the nucleic acids that encode these polypeptides) that include a first portion and a second portion; the first portion includes, e.g., a kinase polypeptide, and the second portion includes an immunoglobulin constant (Fc) region or a detectable marker.

The fusion partner can be, for example, a polypeptide that facilitates secretion, e.g., a secretory sequence. Such a fused polypeptide is typically referred to as a preprotein. The secretory sequence can be cleaved by the host cell to form the mature protein. Also within the invention are nucleic acids that encode a kinase polypeptide fused to a polypeptide sequence to produce an inactive preprotein. Preproteins can be converted into the active form of the protein by removal of the inactivating sequence.

The invention also includes nucleic acids that hybridize, e.g., under stringent hybridization conditions (as defined herein) to all or a portion of the nucleotide sequences represented by SEQ ID NO:1, or its complement. The hybridizing portion of the hybridizing nucleic acids is typically at least 16 (e.g., 20, 30, or 50) nucleotides in length. The hybridizing portion of the hybridizing nucleic acid is at least 50%, e.g., at least 60%, 70%, 80%, 95%, or at least 98% or 100%, identical to the sequence of a portion or all of a nucleic acid encoding a kinase polypeptide or its complement. Hybridizing nucleic acids of the type described herein can be used as a cloning probe, a primer (e.g., a PCR primer), or a diagnostic probe. Nucleic acids that hybridize to the nucleotide sequence represented by SEQ ID NO:1 are considered “antisense oligonucleotides.”

Also useful in the invention are various engineered cells, e.g., transformed host cells, that contain a kinase nucleic acid described herein. A transformed cell is a cell into which (or into an ancestor of which) has been introduced, by means of recombinant DNA techniques, a nucleic acid encoding a kinase polypeptide. Both prokaryotic and eukaryotic cells are included, e.g., fungi, and bacteria, such as E. coli, and the like.

Also useful in the invention are genetic constructs (e.g., vectors and plasmids) that include a nucleic acid of the invention operably linked to a transcription and/or translation sequence to enable expression, e.g., expression vectors. A selected nucleic acid, e.g., a DNA molecule encoding a kinase polypeptide, is “operably linked” when it is positioned adjacent to one or more sequence elements, e.g., a promoter, which direct transcription and/or translation of the sequence such that the sequence elements can control transcription and/or translation of the selected nucleic acid.

The invention also features purified or isolated polypeptides encoded by the C. albicans kinase coding sequence. The terms “protein” and “polypeptide” both refer to any chain of amino acids, regardless of length or post-translational modification (e.g., glycosylation or phosphorylation). Thus, the term kinase polypeptide includes full-length, naturally occurring, isolated kinase proteins, as well as recombinantly or synthetically produced polypeptides that correspond to the full-length, naturally occurring proteins, or to a portion of the naturally occurring or synthetic polypeptide.

A purified or isolated compound is a composition that is at least 60% by weight the compound of interest, e.g., a kinase polypeptide or antibody. Preferably the preparation is at least 75% (e.g., at least 90%, 95%, or even 99%) by weight the compound of interest. Purity can be measured by any appropriate standard method, e.g., column chromatography, polyacrylamide gel electrophoresis, or HPLC analysis.

In the case of polypeptide sequences that are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine.

Where a particular polypeptide is said to have a specific percent identity to a reference polypeptide of a defined length, the percent identity is relative to the reference polypeptide. Thus, a polypeptide that is 50% identical to a reference polypeptide that is 100 amino acids long can be a 50 amino acid polypeptide that is completely identical to a 50 amino acid long portion of the reference polypeptide. It also might be a 100 amino acid long polypeptide that is 50% identical to the reference polypeptide over its entire length. Of course, other polypeptides also will meet the same criteria.

The invention also features purified or isolated antibodies that specifically bind to a C. albicans kinase polypeptide. An antibody “specifically binds” to a particular antigen, e.g., a kinase polypeptide, when it binds to that antigen, but does not recognize and bind to other molecules in a sample, e.g., a biological sample, which naturally includes a kinase polypeptide. In addition, an antibody specifically binds to a C. albicans kinase polypeptide when it does not substantially bind to kinase polypeptides from other genera (e.g., Saccharomyces), particularly kinase polypeptides of an organism to be treated by the methods of the invention (e.g., humans or domesticated animals).

Identifying the Candida albicans Kinase Gene NRK1

The Candida albicans kinase gene is essential for survival. Candida albicans is available from the ATCC. The C. albicans kinase gene was cloned using polymerase chain reaction technology and degenerate primers based on the Saccharomyces cerevisiae kinase gene NRK1 (GenBank Accession No. U00059), which is presumed to be involved in cell morphology and cell wall integrity. The degenerate primers were used to amplify genomic Candida albicans DNA using 35 cycles of: 94° C. for 1 minute, 40° C. for 2 minutes, and 72° C. for 3 minutes. The resulting PCR product was subcloned into the pBluescript cloning vector (Stratagene; La Jolla, Calif.), then sequenced. Based on the resulting sequence, two exact-match primers were created, and the exact-match primers were used to PCR amplify the 5′ and 3′ halves of the NRK1 gene from a Candida albicans cDNA library. The cDNA library was made using the vector pYES2 (Invitrogen; Palo Alto, Calif.). For PCR amplification, one exact-match primer was paired with a primer hybridizing to the 3′ sequence of the multiple cloning site of pYES2. The other exact-match primer was paired with a primer hybridizing to the pGAL sequences in pYES2. PCR amplification of the 5′ and 3′ halves of the kinase gene was carried out with 30 cycles of 94° C. for 30 seconds, 55° C. for 30 seconds, 72° C. for 2.5 minutes. The resulting PCR products were cloned into the pBluescript vector and sequenced to obtain the cDNA sequence of the Candida albicans NRK1 ortholog. The entire kinase open reading frame was subsequently amplified using primers that exactly matched each of (a) the first methionine codon and (b) the stop codon of the kinase open reading frame. The amplified open reading frame subsequently was cloned into the pCRTOPO vector (Invitrogen) using TA cloning methods (Invitrogen).

Identification of Kinase Genes in Additional Fungal Strains

Since a specific CaKinase gene has been identified, this gene, or fragments thereof, can be used to detect homologous genes in yet other organisms. Fragments of a nucleic acid (DNA or RNA) encoding a kinase polypeptide (or sequences complementary thereto) can be used as probes in conventional nucleic acid hybridization assays of various organisms. For example, nucleic acid probes (which typically are 8-30, or usually 15-20, nucleotides in length) can be used to detect kinase genes in art-known molecular biology methods, such as Southern blotting, Northern blotting, dot or slot blotting, PCR amplification methods, colony hybridization methods, and the like. Typically, an oligonucleotide probe based on the nucleic acid sequences described herein, or fragment thereof, is labeled and used to screen a genomic library constructed from mRNA obtained from a fungal strain of interest. A suitable method of labeling involves using polynucleotide kinase to add ³²P-labeled ATP to the oligonucleotide used as the probe. This method is well known in the art, as are several other suitable methods (e.g., biotinylation and enzyme labeling).

Hybridization of the oligonucleotide probe to the library, or other nucleic acid sample, typically is performed under moderate to high stringency conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

High stringency conditions are hybridizing at 68° C. in 5× SSC/5× Denhardt's solution/1.0% SDS, or in 0.5 M NaHPO₄ (pH 7.2)/1 mM EDTA/7% SDS, or in 50% formamide/0.25 M NaHPO₄ (pH 7.2)/0.25 M NaCl/1 mM EDTA/7% SDS; and washing in 0.2× SSC/0.1% SDS at room temperature or at 42° C., or in 0.1× SSC/0.1% SDS at 68° C., or in 40 mM NaHPO₄ (pH 7.2)/1 mM EDTA/5% SDS at 50° C., or in 40 mM NaHPO₄ (pH 7.2) 1 mM EDTA/1% SDS at 50° C. Stringent conditions include washing in 3× SSC at 42° C. The parameters of salt concentration and temperature can be varied to achieve the optimal level of identity between the probe and the target nucleic acid. Additional guidance regarding such conditions is available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2. 10.

In one approach, libraries constructed from pathogenic or non-pathogenic fungal strains are screened. For example, such strains can be screened for expression of the kinase gene of the invention by Northern blot analysis. Upon detection of transcripts of the kinase gene, libraries can be constructed from RNA isolated from the appropriate strain, utilizing standard techniques well known to those of skill in the art. Alternatively, a total genomic DNA library can be screened using a kinase gene probe.

New gene sequences can be isolated, for example, by performing PCR using two degenerate oligonucleotide primer pools designed on the basis of nucleotide sequences within the kinase gene as depicted herein. The template for the reaction can be DNA obtained from strains known or suspected to express the kinase gene of the invention. The PCR product can be subcloned and sequenced.

Synthesis of the various kinase polypeptides (or an antigenic fragment thereof) for use as antigens, or for other purposes, can be accomplished using any of the various art-known techniques. For example, a kinase polypeptide, or an antigenic fragment(s), can be synthesized chemically in vitro, or enzymatically (e.g., by in vitro transcription and translation). Alternatively, the gene can be expressed in, and the polypeptide purified from, a cell (e.g., a cultured cell) by using any of the numerous, available gene expression systems. For example, the polypeptide antigen can be produced in a prokaryotic host (e.g., E. coli) or in eukaryotic cells, such as yeast cells.

Proteins and polypeptides can also be produced in plant cells, if desired. For plant cells, viral expression vectors (e.g., cauliflower mosaic virus and tobacco mosaic virus) and plasmid expression vectors (e.g., Ti plasmid) are suitable. Such cells are available from a wide range of sources (e.g., the American Type Culture Collection, Manassas, Va.; also, see, e.g., Ausubel et al., Current Protocols in Molecular Biology, John Wiley & Sons, New York, 1994). The optimal methods of transformation or transfection and the choice of expression vehicle will depend on the host system selected. Transformation and transfection methods are described, e.g., in Ausubel et al., supra; expression vehicles can be chosen from those provided, e.g., in Cloning Vectors: A Laboratory Manual (P. H. Pouwels et al., 1985, Supp. 1987). The host cells harboring the expression vehicle can be cultured in conventional nutrient media, adapted as needed for activation of a chosen gene, repression of a chosen gene, selection of transformants, or amplification of a chosen gene.

If desired, the kinase polypeptide can be produced as a fusion protein. For example, the expression vector pUR278 (Ruther et al., EMBO J., 2:1791, 1983) can be used to create lacZ fusion proteins. The art-known pGEX vectors can be used to express foreign polypeptides as fusion proteins with glutathione S-transferase (GST). In general, such fusion proteins are soluble and can be easily purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an exemplary expression system, a baculovirus such as Autographa califormica nuclear polyhedrosis virus (AcNPV), which grows in Spodoptera frugiperda cells, can be used as a vector to express foreign genes. A coding sequence encoding a kinase polypeptide can be cloned into a non-essential region (for example the polyhedrin gene) of the viral genome and placed under control of a promoter, e.g., the polyhedrin promoter or an exogenous promoter. Successful insertion of a gene encoding a kinase polypeptide can result in inactivation of the polyhedrin gene and production of non-occluded recombinant virus (i.e., virus lacking the proteinaceous coat encoded by the polyhedrin gene). These recombinant viruses are then typically used to infect insect cells (e.g., Spodopterafrugiperda cells) in which the inserted gene is expressed (see, e.g., Smith et al., J. Virol., 46:584, 19183; Smith, U.S. Pat. No. 4,215,051). If desired, mammalian cells can be used in lieu of insect cells, provided that the virus is engineered such that the gene encoding the kinase polypeptide is placed under the control of a promoter that is active in mammalian cells.

In mammalian host cells, a number of viral-based expression systems can be utilized. When an adenovirus is used as an expression vector, the nucleic acid sequence encoding the kinase polypeptide can be ligated to an adenovirus transcription/translation control complex, e.g., the late promoter and tripartite leader sequence. This chimeric gene can then be inserted into the adenovirus genome by in vitro or in vivo recombination. Insertion into a non-essential region of the viral genome (e.g., region E1 or E3) will result in a recombinant virus that is viable and capable of expressing a kinase gene product in infected hosts (see, e.g., Logan, Proc. Natl. Acad. Sci. USA, 81:3655, 1984).

Specific initiation signals can be required for efficient translation of inserted nucleic acid sequences. These signals include the ATG initiation codon and adjacent sequences. In general, exogenous translational control signals, including, perhaps, the ATG initiation codon, should be provided. Furthermore, the initiation codon must be in phase with the reading frame of the desired coding sequence to ensure translation of the entire sequence. These exogenous translational control signals and initiation codons can be of a variety of origins, both natural and synthetic. The efficiency of expression can be enhanced by the inclusion of appropriate transcription enhancer elements, or transcription terminators (Bittner et al., Methods in Enzymol., 153:516, 1987).

The kinase polypeptide can be expressed individually or as a fusion with a heterologous polypeptide, such as a signal sequence or other polypeptide having a specific cleavage site at the N-and/or C-terminus of the protein or polypeptide. The heterologous signal sequence selected should be one that is recognized and processed, i.e., cleaved by a signal peptidase, by the host cell in which the fusion protein is expressed.

A host cell can be chosen that modulates the expression of the inserted sequences, or modifies and processes the gene product in a specific, desired fashion. Such modifications and processing (e.g., cleavage) of protein products can facilitate optimal functioning of the protein. Various host cells have characteristic and specific mechanisms for post-translational processing and modification of proteins and gene products. Appropriate cell lines or host systems familiar to those of skill in the art of molecular biology can be chosen to ensure the correct modification and processing of the foreign protein expressed. To this end, eukaryotic host cells that possess the cellular machinery for proper processing of the primary transcript, and phosphorylation of the gene product can be used. Such mammalian host cells include, but are not limited to, CHO, VERO, BHK, HeLa, COS, MDCK, 293, 3T3, W138, and choroid plexus cell lines.

If desired, the kinase polypeptide can be produced by a stably-transfected mammalian cell line. A number of vectors suitable for stable transfection of mammalian cells are available to the public, see, e.g., Pouwels et al. (supra); methods for constructing such cell lines are also publicly known, e.g., in Ausubel et al. (supra). In one example, DNA encoding the protein is cloned into an expression vector that includes the dihydrofolate reductase (DHFR) gene. Integration of the plasmid and, therefore, the gene encoding the CaKinase polypeptide into the host cell chromosome is selected for by including 0.01-300 μM methotrexate in the cell culture medium (as described in Ausubel et al., supra). This dominant selection can be accomplished in most cell types.

Recombinant protein expression can be increased by DHFR-mediated amplification of the transfected gene. Methods for selecting cell lines bearing gene amplifications are described in Ausubel et al. (supra); such methods generally involve extended culture in medium containing gradually increasing levels of methotrexate. DHFR-containing expression vectors commonly used for this purpose include pCVSEII-DHFR and pAdD26SV(A) (described in Ausubel et al., supra).

A number of other selection systems can be used, including but not limited to, herpes simplex virus thymidine kinase genes, hypoxanthine-guanine phosphoribosyltransferase genes, and adenine phosphoribosyltransferase genes, which can be employed in tk, hgprt, or aprt cells, respectively. In addition, gpt, which confers resistance to mycophenolic acid (Mulligan et al., Proc. Natl. Acad. Sci. USA, 78:2072, 1981); neo, which confers resistance to the aminoglycoside G-418 (Colberre-Garapin et al., J. Mol. Biol., 150:1, 1981); and hygro, which confers resistance to hygromycin (Santerre et al., Gene, 30:147, 1981), can be used.

Alternatively, any fusion protein can be purified by utilizing an antibody or other molecule that specifically bind to the fusion protein being expressed. For example, a system described in Janknecht et al., Proc. Natl. Acad. Sci. USA, 88:8972 (1981), allows for the ready purification of non-denatured fusion proteins expressed in human cell lines. In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns, and histidine-tagged proteins are selectively eluted with imidazole-containing buffers.

Alternatively, a kinase polypeptide, or a portion thereof, can be fused to an immunoglobulin Fc domain. Such a fusion protein can be purified using a protein A column, for example. Moreover, such fusion proteins permit the production of a chimeric form of a kinase polypeptide having increased stability in vivo.

Once the recombinant kinase polypeptide is expressed, it can be isolated (i.e., purified). Secreted forms of the polypeptides can be isolated from cell culture media, while non-secreted forms must be isolated from the host cells. Polypeptides can be isolated by affinity chromatography. For example, an anti-kinase antibody (e.g., produced as described herein) can be attached to a column and used to isolate the protein. Lysis and fractionation of cells harboring the protein prior to affinity chromatography can be performed by standard methods (see, e.g., Ausubel et al., supra). Alternatively, a fusion protein can be constructed and used to isolate a kinase polypeptide (e.g., a kinase-maltose binding fusion protein, a kinase-β-galactosidase fusion protein, or a kinase-trpE fusion protein; see, e.g., Ausubel et al., supra; New England Biolabs Catalog, Beverly, Mass.). The recombinant protein can, if desired, be further purified, e.g., by high performance liquid chromatography using standard techniques (see, e.g., Fisher, Laboratory Techniques In Biochemistry And Molecular Biology, eds., Work and Burdon, Elsevier, 1980).

Given the amino acid sequences described herein, polypeptides useful in practicing the invention, particularly fragments of CaKinase, can be produced by standard chemical synthesis (e.g., by the methods described in Solid Phase Peptide Synthesis, 2nd ed., The Pierce Chemical Co., Rockford, Ill., 1984) and used as antigens, for example.

Antibodies

The kinase polypeptides (or antigenic fragments or analogs of such polypeptides) can be used to raise antibodies useful in the invention, and such polypeptides can be produced by recombinant or peptide synthetic techniques (see, e.g., Solid Phase Peptide Synthesis, supra; Ausubel et al., supra). In general, the polypeptides can be coupled to a carrier protein, such as KLH, as described in Ausubel et al., supra, mixed with an adjuvant, and injected into a host mammal. A “carrier” is a substance that confers stability on, and/or aids or enhances the transport or immunogenicity of, an associated molecule. Antibodies can be purified, for example, by affinity chromatography methods in which the polypeptide antigen is immobilized on a resin.

In particular, various host animals can be immunized by injection of a polypeptide of interest. Examples of suitable host animals include rabbits, mice, guinea pigs, and rats. Various adjuvants can be used to increase the immunological response, depending on the host species, including but not limited to Freund's (complete and incomplete adjuvant), adjuvant mineral gels such as aluminum hydroxide, surface active substances such as lysolecithin, pluronic polyols, polyanions, peptides, oil emulsions, keyhole limpet hemocyanin, dinitrophenol, BCG (bacille Calmette-Guerin), and Corynebacterium parvum. Polyclonal antibodies are heterogeneous populations of antibody molecules derived from the sera of the immunized animals.

Antibodies useful in the invention include monoclonal antibodies, polyclonal antibodies, humanized or chimeric antibodies, single chain antibodies, Fab fragments, F(ab′)₂ fragments, and molecules produced using a Fab expression library.

Monoclonal antibodies (mAbs), which are homogeneous populations of antibodies to a particular antigen, can be prepared using the kinase, and standard hybridoma technology (see, e.g., Kohler et al., Nature, 256:495, 1975; Kohler et al., Eur. J. Immunol., 6:511, 1976; Kohler et al., Eur. J. Immunol., 6:292, 1976; Hammerling et al., In: Monoclonal Antibodies and T Cell Hybridomas, Elsevier, N.Y., 1981; Ausubel et al., supra).

In particular, monoclonal antibodies can be obtained by any technique that provides for the production of antibody molecules by continuous cell lines in culture, such as those described in Kohler et al., Nature, 256:495, 1975; U.S. Pat. No. 4,376,110; the human B-cell hybridoma technique (Kosbor et al., Immunology Today, 4:72, 1983; Cole et al., Proc. Natl. Acad. Sci. USA, 80:2026, 1983); and the EBV-hybridoma technique (Cole et al., Monoclonal Antibodies and Cancer Therapy, Alan R. Liss, Inc., pp. 77-96, 1983). Such antibodies can be of any immunoglobulin class including IgG, IgM, IgE, IgA, IgD, and any subclass thereof. The hybridomas producing the mAbs of this invention can be cultivated in vitro or in vivo.

Once produced, polyclonal or monoclonal antibodies are tested for specific recognition of a C. albicans kinase in an immunoassay, such as a Western blot or immunoprecipitation analysis using standard techniques, e.g., as described in Ausubel et al., supra. Antibodies that specifically bind to the kinase polypeptide, or conservative variants are useful in the invention. For example, such antibodies can be used in an immunoassay to detect a kinase polypeptide in pathogenic or non-pathogenic strains of fungi.

Preferably, antibodies of the invention are produced using fragments of kinase that appear likely to be antigenic, by criteria such as high frequency of charged residues. In one specific example, such fragments are generated by standard techniques of PCR, and are then cloned into the pGEX expression vector (Ausubel et al., supra). Fusion proteins are expressed in E. coli and purified using a glutathione agarose affinity matrix as described in Ausubel, et al., supra.

If desired, several (e.g., two or three) fusions can be generated for each protein, and each fusion can be injected into at least two rabbits. Antisera can be raised by injections in a series, typically including at least three booster injections. Typically, the antisera is checked for its ability to immunoprecipitate a recombinant kinase polypeptide, or unrelated control proteins, such as glucocorticoid receptor, chloramphenicol acetyltransferase, or luciferase.

Techniques developed for the production of “chimeric antibodies” (Morrison et al., Proc. Natl. Acad. Sci., 81:6851, 1984; Neuberger et al., Nature, 312:604, 1984; Takeda et al., Nature, 314:452, 1984) can be used to splice the genes from a mouse antibody molecule of appropriate antigen specificity together with genes from a human antibody molecule of appropriate biological activity. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. Nos. 4,946,778 and 4,704,692) can be adapted to produce single chain antibodies against a kinase polypeptide. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments can include but are not limited to F(ab′)₂ fragments, which can be produced by pepsin digestion of the antibody molecule, and Fab fragments, which can be generated by reducing the disulfide bridges of F(ab′)₂ fragments. Alternatively, Fab expression libraries can be constructed (Huse et al., Science, 246:1275, 1989) to allow rapid and easy identification of monoclonal Fab fragments with the desired specificity.

Polyclonal and monoclonal antibodies that specifically bind to a kinase polypeptide can be used, for example, to detect expression of kinase in another strain of fungi. For example, a kinase polypeptide can be detected in conventional immunoassays of fungal cells or extracts. Examples of suitable assays include, without limitation, Western blotting, ELISAs, radioimmune assays, and the like.

Assay For Antifungal Agents

The invention provides a method for identifying an antifungal agent. Although the inventor is not bound by any particular theory as to the biological mechanism involved, the new antifungal agents are thought to inhibit specifically (1) the function of the kinase polypeptide or (2) expression of the kinase gene. In preferred methods, screening for potential or candidate antifungal agents is accomplished by identifying those compounds (e.g., small organic molecules) that inhibit the activity of a kinase polypeptide or the expression of a kinase gene. Because kinase is essential for the survival of C. albicans, compounds that inhibit kinase in such assays are expected to be antifungal agents and can be further tested, if desired, in conventional susceptibility assays.

In various suitable methods, screening for antifungal agents is accomplished by (i) identifying those compounds that bind to CaKinase (and are thus candidate antifungal compounds) and (ii) further testing such candidate compounds for their ability to inhibit fungal growth in vitro or in vivo, in which case they are antifungal agents.

Specific binding of a test compound to a polypeptide can be detected, for example, in vitro by reversibly or irreversibly immobilizing the test compound(s) on a substrate, e.g., the surface of a well of a 96-well polystyrene microtitre plate. Methods for immobilizing polypeptides and other small molecules are well known in the art. For example, the microtitre plates can be coated with a kinase polypeptide by adding the polypeptide in a solution (typically, at a concentration of 0.05 to 1 mg/ml in a volume of 1-100 μl) to each well, and incubating the plates at room temperature to 37° C. for 0.1 to 36 hours. Polypeptides that are not bound to the plate can be removed by shaking the excess solution from the plate, and then washing the plate (once or repeatedly) with water or a buffer. Typically, the polypeptide is in water or a buffer. The plate is then washed with a buffer that lacks the bound polypeptide. To block the free protein-binding sites on the plates, the plates are blocked with a protein that is unrelated to the bound polypeptide. For example, 300 μl of bovine serum albumin (BSA) at a concentration of 2 mg/ml in Tris-HCl is suitable. Suitable substrates include those substrates that contain a defined cross-linking chemistry (e.g., plastic substrates, such as polystyrene, styrene, or polypropylene substrates from Corning Costar Corp., Cambridge, Mass., for example). If desired, a beaded particle, e.g., beaded agarose or beaded Sepharose, can be used as the substrate. The kinase is then added to the coated plate and allowed to bind to the test compound (e.g., at 37° C. for 0.5-12 hours). The plate then is rinsed as described above.

Binding of the test compound to the kinase can be detected by any of a variety of art-known methods. For example, an antibody that specifically binds to a kinase polypeptide can be used in an immunoassay. If desired, the antibody can be labeled (e.g., fluorescently or with a radioisotope) and detected directly (see, e.g., West and McMahon, J. Cell Biol. 74:264, 1977). Alternatively, a second antibody can be used for detection (e.g., a labeled antibody that binds to the Fc portion of an anti-YphC antibody). In an alternative detection method, the kinase polypeptide is labeled, and the label is detected (e.g., by labeling a kinase polypeptide with a radioisotope, fluorophore, chromophore, or the like). In still another method, the kinase polypeptide is produced as a fusion protein with a protein that can be detected optically, e.g., using green fluorescent protein (which can be detected under UV light). In an alternative method, the polypeptide can be produced as a fusion protein with an enzyme having a detectable enzymatic activity, such as horseradish peroxidase, alkaline phosphatase, β-galactosidase, or glucose oxidase. Genes encoding all of these enzymes have been cloned and are available for use by those of skill in the art. If desired, the fusion protein can include an antigen, and such an antigen can be detected and measured with a polyclonal or monoclonal antibody using conventional methods. Suitable antigens include enzymes (e.g., horseradish peroxidase, alkaline phosphatase, and β-galactosidase) and non-enzymatic polypeptides (e.g., serum proteins, such as BSA and globulins, and milk proteins, such as caseins).

In various in vivo methods for identifying polypeptides that bind to kinase, the conventional two-hybrid assays of protein/protein interactions can be used (see e.g., Chien et al., Proc. Natl. Acad. Sci. USA, 88:9578, 1991; Fields et al., U.S. Pat. No. 5,283,173; Fields and Song, Nature, 340:245, 1989; Le Douarin et al., Nucleic Acids Research, 23:876, 1995; Vidal et al., Proc. Natl. Acad. Sci. USA, 93:10315-10320, 1996; and White, Proc. Natl. Acad. Sci. USA, 93:10001-10003, 1996). Generally, the two-hybrid methods involve in vivo reconstitution of two separable domains of a transcription factor. One fusion protein contains the kinase polypeptide fused to either a transactivator domain or DNA binding domain of a transcription factor (e.g., of Ga14). The other fusion protein contains a test polypeptide fused to either the DNA binding domain or a transactivator domain of a transcription factor. Once brought together in a single cell (e.g., a yeast cell or mammalian cell), one of the fusion proteins contains the transactivator domain and the other fusion protein contains the DNA binding domain. Therefore, binding of the kinase polypeptide to the test polypeptide (i.e., candidate antifungal agent) reconstitutes the transcription factor. Reconstitution of the transcription factor can be detected by detecting expression of a gene (i.e., a reporter gene) that is operably linked to a DNA sequence that is bound by the DNA binding domain of the transcription factor. Kits for practicing various two-hybrid methods are commercially available (e.g., from Clontech; Palo Alto, Calif.).

The methods described above can be used for high throughput screening of numerous test compounds to identify candidate antifungal (or anti-fungal) agents. Having identified a test compound as a candidate antifungal agent, the candidate antifungal agent can be further tested for inhibition of fungal growth in vitro or in vivo (e.g., using an animal, e.g., rodent, model system) if desired. Using other, art-known variations of such methods, one can test the ability of a nucleic acid (e.g., DNA or RNA) used as the test compound to bind to the kinase.

In vitro, further testing can be accomplished by means known to those in the art such as an enzyme inhibition assay or a whole-cell fungal growth inhibition assay. For example, an agar dilution assay identifies a substance that inhibits fungal growth. Microtiter plates are prepared with serial dilutions of the test compound, adding to the preparation a given amount of growth substrate, and providing a preparation of fungi. Inhibition of fungal growth is determined, for example, by observing changes in optical densities of the fungal cultures.

Inhibition of fungal growth is demonstrated, for example, by comparing (in the presence and absence of a test compound) the rate of growth or the absolute growth of fungal cells. Inhibition includes a reduction in the rate of growth or absolute growth by at least 20%. Particularly potent test compounds can further reduce the growth rate (e.g., by at least 25%, 30%, 40%, 50%, 75%, 80%, or 90%).

Animal (e.g., rodent such as murine) models of fungal infections are known to those of skill in the art, and such animal model systems are acceptable for screening antifungal agents as an indication of their therapeutic efficacy in human patients. In a typical in vivo assay, an animal is infected with a pathogenic strain of fungi, e.g., by inhalation of fungi, and conventional methods and criteria are used to diagnose the mammal as being afflicted with a fungal infection. The candidate antifungal agent then is administered to the mammal at a dosage of 1-100 mg/kg of body weight, and the mammal is monitored for signs of amelioration of disease. Alternatively, the test compound can be administered to the mammal prior to infecting the mammal with the fungi, and the ability of the treated mammal to resist infection is measured. Of course, the results obtained in the presence of the test compound should be compared with results in control animals, which are not treated with the test compound. Administration of candidate antifungal agents to the mammal can be carried out as described below, for example.

Pharmaceutical Formulations

Treatment includes administering a pharmaceutically effective amount of a composition containing an antifungal agent to a subject in need of such treatment, thereby inhibiting or reducing fungal growth in the subject. Such a composition typically contains from about 0.1 to 90% by weight (such as 1 to 20% or 1 to 10%) of an antifungal agent of the invention in a pharmaceutically acceptable carrier.

Solid formulations of the compositions for oral administration can contain suitable carriers or excipients, such as cornstarch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, micro-crystalline cellulose, corn starch, sodium starch glycolate and alginic acid. Tablet binders that can be used include acacia, methylcellulose, sodium carboxymethylcellulose, polyvinylpyrrolidone (Povidone®), hydroxypropyl methylcellulose, sucrose, starch, and ethylcellulose. Lubricants that can be used include magnesium stearates, stearic acid, silicone fluid, talc, waxes, oils, and colloidal silica.

Liquid formulations of the compositions for oral administration prepared in water or other aqueous vehicles can contain various suspending agents such as methylcellulose, alginates, tragacanth, pectin, kelgin, carageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). For intravenous injections, water-soluble versions of the compounds can be administered by the drip method, whereby a pharmaceutical formulation containing the antifungal agent and a physiologically acceptable excipient is infused. Physiologically acceptable excipients can include, for example, 5% dextrose, 0.9% saline, Ringer's solution, or other suitable excipients. For intramuscular preparations, a sterile formulation of a suitable soluble salt form of the compounds can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically acceptable oil base, such as an ester of a long chain fatty acid, (e.g., ethyl oleate).

A topical semi-solid ointment formulation typically contains a concentration of the active ingredient from about 1 to 20%, e.g., 5 to 10% in a carrier such as a pharmaceutical cream base. Various formulations for topical use include drops, tinctures, lotions, creams, solutions, and ointments containing the active ingredient and various supports and vehicles.

The optimal percentage of the antifungal agent in each pharmaceutical formulation varies according to the formulation itself and the therapeutic effect desired in the specific pathologies and correlated therapeutic regimens. Appropriate dosages of the antifungal agents can be determined by those of ordinary skill in the art of medicine by monitoring the mammal for signs of disease amelioration or inhibition, and increasing or decreasing the dosage and/or frequency of treatment as desired. The optimal amount of the antifungal compound used for treatment of conditions caused by or contributed to by fungal infection depends upon the manner of administration, the age and the body weight of the subject, and the condition of the subject to be treated. Generally, the antifungal compound is administered at a dosage of 1 to 100 mg/kg body weight, and typically at a dosage of 1 to 10 mg/kg body weight.

### Other Embodiments

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims. For example, other art-known assays to detect interactions of test compounds with proteins, or to detect inhibition of fungal growth also can be used with the kinase gene.

What is claimed is:

1. An isolated nucleic acid molecule selected from the group consisting of:
(a) a nucleic acid molecule that encodes a polypeptide comprising the amino acid sequence of SEQ ID NO:2;
(b) a nucleic acid molecule that encodes a polypeptide comprising at least 17 contiguous amino acids of SEQ ID NO:2; and
(c) a nucleic acid molecule that encodes a naturally occurring allelic variant of a polypeptide comprising the amino acid sequence of SEQ ID NO:2, wherein the nucleic acid molecule hybridizes under stringent conditions to a nucleic acid molecule consisting of the nucleotide sequence of SEQ ID NO:1, or the complement of SEQ ID NO:1.

2. An isolated nucleic acid molecule selected from the group consisting of:
(a) a nucleic acid molecule comprising the nucleotide sequence of SEQ ID NO:1;
(b) a nucleic acid molecule comprising the nucleotide sequence of SEQ ID NO:1, wherein the “T”s are replaced with “U”s;
(c) a nucleic acid molecule that is complementary to (a) or (b); and
(d) fragments of (a), (b), or (c) that comprise at least 50 contiguous nucleotides of SEQ ID NO:1 or the complement of SEQ ID NO:1.

3. An isolated nucleic acid molecule selected from the group consisting of:
(a) a nucleic acid molecule comprising a nucleotide sequence which is at least about 45% identical to the nucleotide sequence of SEQ ID NO:1 or a complement thereof, wherein the percent identity is calculated using the GAP program in the GCG software package, using a gap weight of 5.000 and a length weight of 0.100;
(b) a nucleic acid molecule comprising a nucleotide sequence that hybridizes to a nucleic acid molecule consisting of the nucleotide sequence of SEQ ID NO:1 under stringent conditions, or a complement thereof; and
(c) a nucleic acid molecule comprising a nucleotide sequence that hybridizes under stringent conditions to a nucleic acid molecule consisting of the nucleotide sequence of the cDNA insert of a plasmid deposited with the ATCC as Accession Number ______ or a complement thereof.

4. A nucleic acid molecule of claim 1, further comprising a vector nucleic acid sequence.

5. A nucleic acid molecule of claim 1, further comprising a nucleic acid sequence encoding a heterologous polypeptide.

6. A host cell that contains the nucleic acid molecule of claim 1.

7. A host cell of claim 6, wherein the cell is a mammalian host cell.

8. A host cell of claim 6, wherein the cell is a non-mammalian host cell.

9. An isolated polypeptide selected from the group consisting of:
(a) a polypeptide comprising a sequence of at least 17 contiguous amino acids of SEQ ID NO:2;
(b) a naturally occurring allelic variant of a polypeptide comprising the amino acid sequence of SEQ ID NO:2 or an amino acid sequence encoded by the cDNA insert of the plasmid deposited with ATCC as Accession Number ______, wherein the polypeptide is encoded by a nucleic acid molecule that hybridizes under stringent conditions to the complement of a nucleic acid molecule consisting of the nucleotide sequence of SEQ ID NO:1; and
(c) a polypeptide encoded by a nucleic acid molecule comprising a nucleotide sequence that is at least 50% identical to SEQ ID NO:1, wherein the percent identity is calculated using the GAP program in the GCG software package, using a gap weight of 5.000 and a length weight of 0.100.

10. A polypeptide of claim 9, further comprising a heterologous amino acid sequence.

11. An antibody that selectively binds to a polypeptide of claim 9.

12. A method for producing a polypeptide, the method comprising culturing the host cell of claim 6 under conditions in which the nucleic acid molecule is expressed, wherein the polypeptide is selected from the group consisting of:
(a) a polypeptide comprising the amino acid sequence of SEQ ID NO:2;
(b) a polypeptide comprising at least 17 contiguous amino acids of SEQ ID NO:2; and
(c) a naturally occurring allelic variant of a polypeptide comprising the amino acid sequence of SEQ ID NO:2, wherein the polypeptide is encoded by a nucleic acid molecule that hybridizes under stringent conditions to a nucleic acid molecule comprising SEQ ID NO:1.

13. A method for identifying a candidate antifungal agent, the method comprising:
(a) obtaining a first cell and a second cell, the first and second cells being capable of expressing NRK1;
(b) contacting the first cell with a test compound;
(c) determining a level of expression of NRK1 in the first cell and second cells; and
(d) comparing the level of expression in the first cell with the level of expression in the second cell;
wherein a level of expression of NRK1 in the first cell less than the level of expression of NRK1 in the second cell indicates that the test compound is a candidate antifungal agent; and
wherein NRK1 is a first nucleic acid molecule that encodes a polypeptide comprising the amino acid sequence of SEQ ID NO:2 or a naturally occurring allelic variant of a polypeptide comprising the amino acid sequence of SEQ ID NO:2, and wherein the first nucleic acid molecule hybridizes to a second nucleic acid molecule under stringent conditions, the second nucleic acid molecule consisting of the nucleotide sequence of SEQ ID NO:1 or the complement of SEQ ID NO:1.

14. A method of claim 13, wherein the level of expression is measured by measuring the amount of NRK1 mRNA in the cell.

15. A method of claim 13, wherein the level of expression is measured by measuring the amount of protein encoded by NRK1.

16. A method for identifying a candidate antifungal agent for the treatment of a fungal infection, the method comprising
(a) obtaining a first cell and a second cell, the first and second cells being capable of expressing NRK1;
(b) contacting the first cell with a test compound;
(c) determining a level activity of a polypeptide encoded by NRK1 in the first and second cells; and
(d) comparing the level of activity of the polypeptide in the first cell with the level of activity of the polypeptide in the second cell;
wherein a level of activity of the polypeptide encoded by NRK1 in the first cell less than a level of activity of the polypeptide encoded by NRK1 in the second cell indicates that the compound is a candidate antifungal agent;
wherein NRK1 is a first nucleic acid molecule that encodes a polypeptide comprising the amino acid sequence of SEQ ID NO:2 or a naturally occurring allelic variant of a polypeptide comprising the amino acid sequence of SEQ ID NO:2, and wherein the first nucleic acid molecule hybridizes to a second nucleic acid molecule under stringent conditions, the second nucleic acid molecule consisting of the nucleotide sequence of SEQ ID NO:1 or the complement of SEQ ID NO:1.

17. A method for identifying a candidate antifungal agent for the treatment of a fungal infection, the method comprising
(a) obtaining a first sample of cells and a second sample of cells, the first and second samples of cells being capable of expressing NRK1 in the presence of a test compound;
(b) contacting the first sample of cells with a test compound; and
(c) comparing the growth of the first sample of cells with the growth of the second sample of cells;
wherein growth of the first sample of cells slower than growth of the second sample of cells indicates the test compound is a candidate antifungal agent; and
wherein NRK1 is a first nucleic acid molecule that encodes a polypeptide comprising the amino acid sequence of SEQ ID NO:2 or a naturally occurring allelic variant of a polypeptide comprising the amino acid sequence of SEQ ID NO:2, wherein the first nucleic acid molecule hybridizes to a second nucleic acid molecule under stringent conditions, the second nucleic acid molecule consisting of the nucleotide sequence of SEQ ID NO:1 or the complement of SEQ ID NO:1.

18. A method of claim 17, wherein the first and second samples of cells comprise fungal cells.

19. A method of treating a fungal infection in a patient, the method comprising administering to the patient an effective amount of an antifungal agent identified using the method of claim 13.

20. A method of claim 19, wherein the antifungal agent is selected from the group consisting of a polypeptide, ribonucleic acid, small molecule, and deoxyribonucleic acid.

21. A method of claim 19, wherein the antifungal agent is an antisense oligonucleotide.

22. A method of claim 19, wherein the antifungal agent is a ribozyme.

23. A method for identifying a candidate antifungal agent useful for treating a fungal infection, the method comprising
(a) contacting a polypeptide encoded by NRK1 with a test compound; and
(b) detecting binding of the test compound to the polypeptide, wherein a compound that binds to the NRK1 polypeptide indicates that the compound is a candidate antifungal agent, and wherein the polypeptide is encoded by a gene selected from the group consisting of a first nucleic acid molecule that encodes a polypeptide comprising the amino acid sequence of SEQ ID NO:2 or a naturally occurring allelic variant of a polypeptide comprising the amino acid sequence of SEQ ID NO:2, and wherein the first nucleic acid molecule hybridizes to a second nucleic acid molecule under stringent conditions, the second nucleic acid molecule consisting of the nucleotide sequence of SEQ ID NO:1 or the complement of SEQ ID NO:1.

24. The method of claim 23, further comprising:
determining whether the candidate compound that binds to the NRK1 polypeptide inhibits growth of fungi, relative to growth of fungi grown in the absence of the test compound, wherein inhibition of growth indicates that the candidate compound is an antifungal agent.

25. A method of claim 23, wherein the test compound is immobilized on a substrate, and binding of the test compound to the NRK1 polypeptide is detected as immobilization of the NRK1 polypeptide on the immobilized test compound.

26. A method of claim 25, wherein immobilization of the NRK1 polypeptide on the test compound is detected in an immunoassay with an antibody that specifically binds to the NRK1 polypeptide.

27. A method of claim 23, wherein the test compound is selected from the group consisting of a polypeptide, ribonucleic acid, small molecule, and deoxyribonucleic acid.

28. A method of claim 23, wherein:
the NRK1 polypeptide is provided as a first fusion protein comprising a NRK1 polypeptide fused to (i) a transcription activation domain of a transcription factor or (ii) a DNA-binding domain of a transcription factor; and the test compound is a polypeptide that is provided as a second fusion protein comprising the test compound fused to (i) a transcription activation domain of a transcription factor or (ii) a DNA-binding domain of a transcription factor, to interact with the first fusion protein; and
binding of the test compound to the NRK1 polypeptide is detected as reconstitution of a transcription factor.

29. A pharmaceutical formulation for the treatment of a fungal infection, the formulation comprising an antifungal agent identified by the method of claim 23 and a pharmaceutically acceptable excipient.

30. A method for treating an organism having a fungal infection, the method comprising administering to the organism a therapeutically effective amount of the pharmaceutical formulation of claim 29.

31. A method of claim 30, wherein the organism is a human.

32. A method of treating an antifungal infection in an organism, the method comprising administering to the organism a therapeutically effective amount of the antibody of claim 11.

33. A method of claim 32, wherein the antibody is a monoclonal antibody.

34. A pharmaceutical formulation for the treatment of a fungal infection in an organism, the formulation comprising a ribozyme of claim 22 and a pharmaceutically acceptable excipient.

35. A pharmaceutical formulation for the treatment of a fungal infection in an organism, the formulation comprising an antisense nucleic acid of claim 21 and a pharmaceutically acceptable excipient.

36. A method for identifying a candidate compound for treating a fungal infection, the method comprising:
(a) contacting a NRK1 polynucleotide with a test compound; and
(b) detecting binding of the test compound to the NRK1 polynucleotide, wherein a compound that binds to the NRK1 polynucleotide is a candidate compound for treating a fungal infection, and wherein the NRK1 polynucleotide is selected from the group consisting of
(i) a nucleic acid molecule that encodes a polypeptide comprising the amino acid sequence of SEQ ID NO:2; and
(ii) a nucleic acid molecule that encodes a naturally occurring allelic variant of a polypeptide comprising the amino acid sequence of SEQ ID NO:2, wherein the nucleic acid molecule hybridizes under stringent conditions to a nucleic acid molecule consisting of the nucleotide sequence of SEQ ID NO:1 or the complement of SEQ ID NO:1.

37. The method of claim 36, further comprising:
determining whether a candidate compound that binds to the NRK1 polynucleotide inhibits growth of fungi, relative to growth of fungi grown in the absence of the test compound, wherein inhibition of growth indicates that the candidate compound is an antifungal compound.

38. The method of claim 36, wherein the test compound is selected from the group consisting of a polypeptide, small molecule, ribonucleic acid, and deoxyribonucleic acid.

39. The method of claim 36, wherein the test compound is an antisense oligonucleotide.

40. The method of claim 36, wherein the test compound is a ribozyme.

41. A method for identifying a candidate compound for treating a fungal infection, the method comprising:
(a) contacting a homolog of NRK1 with a test compound; and
(b) detecting binding of the test compound to the homolog of NRK1, wherein a compound that binds to the homolog of NRK1 is a candidate compound for treating a fungal infection, wherein NRK1 is selected from the group consisting of a first nucleic acid molecule that encodes either a polypeptide comprising the amino acid sequence of SEQ ID NO:2 or a naturally occurring allelic variant of a polypeptide comprising the amino acid sequence of SEQ ID NO:2, wherein the first nucleic acid molecule hybridizes to a second nucleic acid molecule under stringent conditions, the second nucleic acid molecule consisting of the nucleotide sequence of SEQ ID NO:1 or the complement of SEQ ID NO:1.

42. A method of claim 41, further comprising:
determining whether a candidate compound that binds to the homolog of NRK1 inhibits growth of fungi, relative to growth of fungi grown in the absence of the test compound that binds to the homolog of NRK1, wherein inhibition of growth indicates that the candidate compound is an antifungal agent.

43. A method of claim 41, wherein the homolog of NRK1 is derived from a non-pathogenic fungus.

44. A method of claim 41, wherein the homolog of NRK1 is derived from a pathogenic fungus.

45. A method of claim 41, wherein the test compound is immobilized on a substrate, and binding of the test compound to the homolog of NRK1 is detected as immobilization of the homolog of NRK1 on the immobilized test compound.

46. The method of claim 45, wherein immobilization of the homolog of NRK1 on the test compound is detected in an immunoassay with an antibody that specifically binds to the homolog of NRK1.

47. The method of claim 41, wherein the test compound is selected from the group consisting of a polypeptide, ribonucleic acid, small molecule, and deoxyribonucleic acid.
